SEQUENCE LISTING (36 sequences)

SEQ ID NO: 1 - DNA, 20 bp, Homo sapiens
gttcgacagtcagccgcatc

SEQ ID NO: 2 - DNA, 19 bp, Homo sapiens
ggaatttgccatgggtgga

SEQ ID NO: 3 - DNA, 22 bp, homo sapiens
accaggcgcccaatacgaccaa

SEQ ID NO: 4 - DNA, 21 bp, homo sapiens
gccaggatctgtggtggtaca

SEQ ID NO: 5 - DNA, 20 bp, homo sapiens
ctccacgtcgtggacattga

SEQ ID NO: 6 - DNA, 26 bp, homo sapiens
ggcacatcactcacgctgacgtctga

SEQ ID NO: 7 - DNA, 21 bp, homo sapiens
cccaactttgatgccagcatt

SEQ ID NO: 8 - DNA, 19 bp, homo sapiens
cagcatccattgggcatga

SEQ ID NO: 9 - DNA, 21 bp, homo sapiens
tgtgatggagcccgggatgca

SEQ ID NO: 10 - DNA, 21 bp, homo sapiens
cgaaaacagcccactgatgtc

SEQ ID NO: 11 - DNA, 20 bp, homo sapiens
tggaggcctgagttggaatt

SEQ ID NO: 12 - DNA, 25 bp, homo sapiens
agggcgatacctctcccacactggc

SEQ ID NO: 13 - DNA, 19 bp, homo sapiens
tactcgctcgagggcaaca

SEQ ID NO: 14 - DNA, 20 bp, homo sapiens
tgcagtgcagggtcacattc

SEQ ID NO: 15 - DNA, 20 bp, homo sapiens
ccaggagctgcggacctcgc

SEQ ID NO: 16 - DNA, 23 bp, homo sapiens
gtgtgggtggtctctggagtaga

SEQ ID NO: 17 - DNA, 24 bp, homo sapiens
aaatccacagctaccagctttaca

SEQ ID NO: 18 - DNA, 22 bp, homo sapiens
tggaccgtcccagcacagacca

SEQ ID NO: 19 - DNA, 19 bp, homo sapiens
ccacttctgcctccatcca

SEQ ID NO: 20 - DNA, 19 bp, homo sapiens
ggccttgagcgttgttggt

SEQ ID NO: 21 - DNA, 22 bp, homo sapiens
tcaacgccaacaggcaccgttc

SEQ ID NO: 22 - DNA, 21 bp, homo sapiens
gcaaggtggctttgtagtgga

SEQ ID NO: 23 - DNA, 24 bp, homo sapiens
agactgtatttccaagtgccaacc

SEQ ID NO: 24 - DNA, 28 bp, homo sapiens
acaccaaaatggcctacctccaaatccc

SEQ ID NO: 25 - DNA, 20 bp, homo sapiens
gaggccgagtgtttgtccat

SEQ ID NO: 26 - DNA, 27 bp, homo sapiens
ccatttatgtagaaaggtttgtcattg

SEQ ID NO: 27 - DNA, 19 bp, homo sapiens
tgggcccacctccgttggg

SEQ ID NO: 28 - DNA, 24 bp, homo sapiens
cctgaagtcacagatgatgtggaa

SEQ ID NO: 29 - DNA, 24 bp, homo sapiens
aggcactcctgatacagtaaacga

SEQ ID NO: 30 - DNA, 28 bp, homo sapiens
cagacgacttcagcagactcacctctgg

SEQ ID NO: 31 - DNA, 23 bp, homo sapiens
gaatcaagcagcaaaacatttcc

SEQ ID NO: 32 - DNA, 25 bp, homo sapiens
gtggcagtactatcaaagtgaacca

SEQ ID NO: 33 - DNA, 28 bp, homo sapiens
cccaggatccattttcaacagtctgcag

SEQ ID NO: 34 - DNA, 23 bp, mouse
gtcccgtagacaaaatggtgaag

SEQ ID NO: 35 - DNA, 17 bp, mouse
gtgaccaggcgcccaat

SEQ ID NO: 36 - DNA, 21 bp, mouse
cggtgtgaacggatttggccg

(19) United States
(12) Patent Application Publication
(10) Pub. No.: US 2005/0096258 A1
(43) Pub. Date: May 5, 2005

(54) Methods and compositions for treating gastrointestinal tract mucin production associated disease conditions

(76) Inventors: Andrew Grupe, Redwood City, CA (US); David Edmund Szymkowski, Mountain View, CA (US)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 10/967,463

(22) Filed: Oct. 18, 2004

Related U.S. Application Data
(62) Division of application No. 09/925,547, filed on Aug. 8, 2001.
(60) Provisional application No. 60/161,483, filed on Aug. 9, 2000.

(51) Int. Cl. A61K 38/17; A61K 31/44

(52) U.S. Cl. 514/2; 514/355

(57) ABSTRACT

The invention provides a method of treatment for an animal having a disease state associated with a mucus secretion disorder with a chloride channel modulator. The invention further provides for a method of screening for therapeutic compounds using the chloride channel. Also provided is a transgenic animal having a disrupted chloride gene, and a method of diagnosing a disease state associated with a mucus secretion disorder.

### CROSS-REFERENCE

This application claims the benefit of priority of U.S. Provisional Patent Application Ser. No. 60/161,483 filed Aug. 9, 2000, which is incorporated herein by reference in its entirety.

### FIELD OF THE INVENTION

The present invention relates to a method of treating or preventing a disease state associated with a pulmonary or gastrointestinal disorder using a chloride channel modulator. Also provided is a method of screening for therapeutic compounds using the chloride channel.

### BACKGROUND OF THE INVENTION

Mucus is a thin film of protective viscoelastic liquid which lines the airways, gastrointestinal tract, and other organs containing mucus membranes. Mucus is an aqueous solution in which the major component is a glycoconjugate, known as mucin. Mucin secretion may be constitutive, regulated, or in response to external stimuli, in particular irritants.

Chloride channels, widely distributed throughout various tissues, play roles as diverse as maintaining membrane potential in muscles to movement of Cl⁻ for fluid and electrolyte transport in epithelial tissues. Several studies have suggested that a calcium activated secretory pathway for Cl⁻ may play an important role in modulating the disease severity in various tissues of CF patients. (See, Anderson and Welsh (1991) Proc. Natl. Acad. Sci. 88:6003-6007; Knowles, et al. (1991) N. Engl. J. Med. 325:533-538; Mason, et al. (1991) Br. J. Pharmocol. 103:1649-1656; Wagner, et al. (1991) Nature 349:793-796; and Willumsen and Boucher (1989) Am. J. Physiol. 256(Cell Physiol. 25):C226-C235.)

A new family of proteins has recently been discovered that mediates a Ca²⁺-activated Cl⁻ conductance in a variety of tissues. Six members of this family have been identified: bovine lung endothelial cell adhesion molecule, Lu-ECAM-1 (Elble, et al. (1997) J. Biol. Chem. 272:27853-27861); bovine Ca²⁺-activated Cl⁻, CaCC or bCLCA1 (Cunningham, et al. (1995) J. Biol. Chem. 270:31016-31026); murine CLCA1, mCLCA1 (Gandhi, et al. (1998) J. Biol. Chem. 273:32096-32101); human CLCA1, hCLCA1 (Gruber, et al. (1998) Genomics 54:200-214); murine Gob-5, mGob-5 (Komiya, et al. (1999) Biochem. Biophys. Res. Comm. 255:347-351; and human CLCA2, hCLCA2 (Gruber, et al. (1999) Am. J. Physiol. 276(Cell Physiol. 45):C1261-C1270.) Recently, Holroyd, et al., PCT publication No. WO 99/44620, described a calcium activated chloride channel that is induced by IL-9. Collectively, these channels are referred to as Calcium-activated Chloride Channels (CaCC).

It has been reported that the stimulation of chloride secretion results in the secretion of mucin from goblet cells in the intestinal epithelium. (Halm, et al. (1995) Am. J. Physiol. 269:929-942.) The murine Gob-5 gene, mGob-5, has been shown, through in situ hybridization, to be expressed in the mucus-secreting cells of the stomach, small intestine, colon, and uterus, along with slight expression in the trachea. (Komiya, et al. supra.) Similarly it was suggested that human CLCA1, the human homolog of mGob-5, may also have a role in mucus secretion, however, previous studies found expression of this chloride channel restricted to the small intestine and colon mucosa. (See, Gruber, et al. (1998) supra.)

Presently, there is a lack of knowledge regarding expression of CaCC in the intestines and its precise connection to mucus hypersecretion. The present invention fills this need by causally linking this chloride channel to mucin gene expression and improper mucus secretion in the gastrointestinal tract. Thus modulators of CaCC are useful in the treatment of disorders associated with improper mucus secretion.

Literature

Corfield and Warren (1996) J. Pathol. 180:8-17; Ryder et al. (1995) Digestion 56 :370-376 ; Pullan et al. (1994) Gut 35 :353-359.

### SUMMARY OF THE INVENTION

The present invention is based, in part, upon the discovery of the causal link between expression of a Ca²⁺-activated Cl⁻ channel (CaCC), in particular human CLCA1 (hCLCA1) and improper mucus secretion, particularly in the gastrointestinal tract.

The present invention provides a method of treating a subject having a disease state associated with a mucus secretion disorder comprising administering to the subject an effective amount of a chloride channel modulator.

In a further embodiment the animal is a mammal, preferably a human. In another embodiment the disease state is associated with mucus hypersecretion in the gastrointestinal tract, e.g., inflammatory bowel syndrome and ulcerative colitis. In further embodiment, the disease state is associated with mucus hyposecretion in the gastrointestinal tract, e.g., Crohn syndrome.

In another embodiment, the chloride channel modulator is a Ca²⁺-activated Cl⁻ channel (CaCC) modulator, preferably human CLCA1 (hCLCA1) modulator. In a further embodiment, the modulator can be an antagonist or an agonist.

The present invention also provides a method of screening for a compound that modulates CaCC activity comprising contacting a CaCC, or fragment thereof with the compound; and detecting modulation of CaCC activity. In another embodiment, the CaCC is expressed on a cell or tissue; or immobilized on a solid support. In a further embodiment, the compound is an antagonist of CaCC activity; or an agonist of CaCC activity. In still a further embodiment, CaCC is hCLCA1.

Also provided is a compound identified by contacting a CaCC, or fragment thereof with the compound; and detecting modulation of CaCC activity. In another embodiment, the compound is in conjunction with a pharmaceutically acceptable carrier.

The present invention provides a transgenic nonhuman animal comprising an altered CaCC gene. The transgenic nonhuman animal can underexpress CaCC or overexpress CaCC. Preferably the transgenic animal is a mouse.

Also provided is a method of detecting the presence of a CaCC in a biological sample from a subject suspected of having a disease state associated with a mucus secretion disorder comprising contacting the biological sample containing nucleic acid material with a polynucleotide encoding a CaCC polypeptide under conditions sufficient to form a hybridization complex; detecting the hybridization complex, wherein the presence of the complex correlates with the presence of the polynucleotide encoding CaCC polypeptide.

These and other objects, advantages, and features of the invention will become apparent to those persons skilled in the art upon reading the details of the invention as more fully described below.

### BRIEF DESCRIPTION OF THE DRAWING

FIG. 1 shows that mGOB5 (the mouse homolog of hCLCA1) expression is induced in mouse lungs after superdex bead instillation in mouse lung. mGob5 induction is similar to mMUC5AC induction in the same model (see FIG. 3).

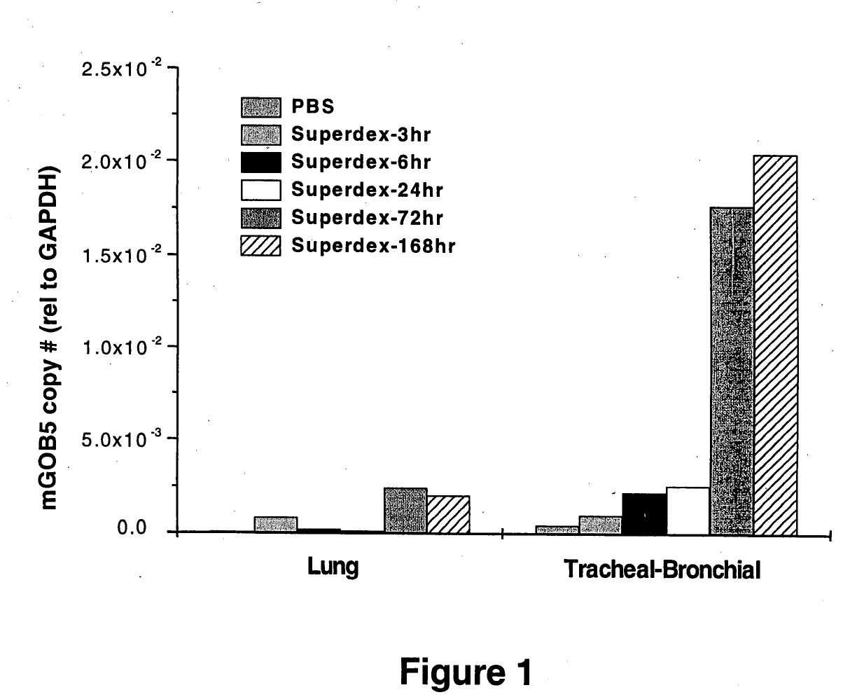

FIGS. 2A and 2B show mCLCA1 and mGob5 expression, respectively, in mouse lungs after superdex bead instillation. Unlike mGob5, mCLCA1 is not induced in mouse lung by superdex bead instillation.

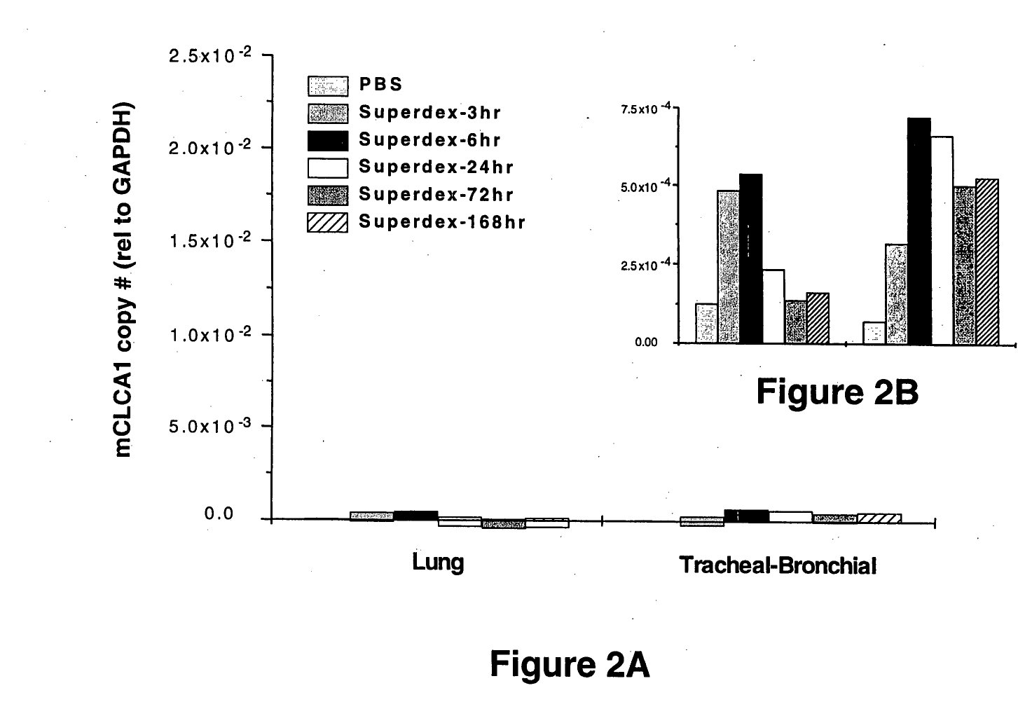

FIG. 3 shows MUC5AC mucin gene expression is induced by superdex bead instillation in mouse lung.

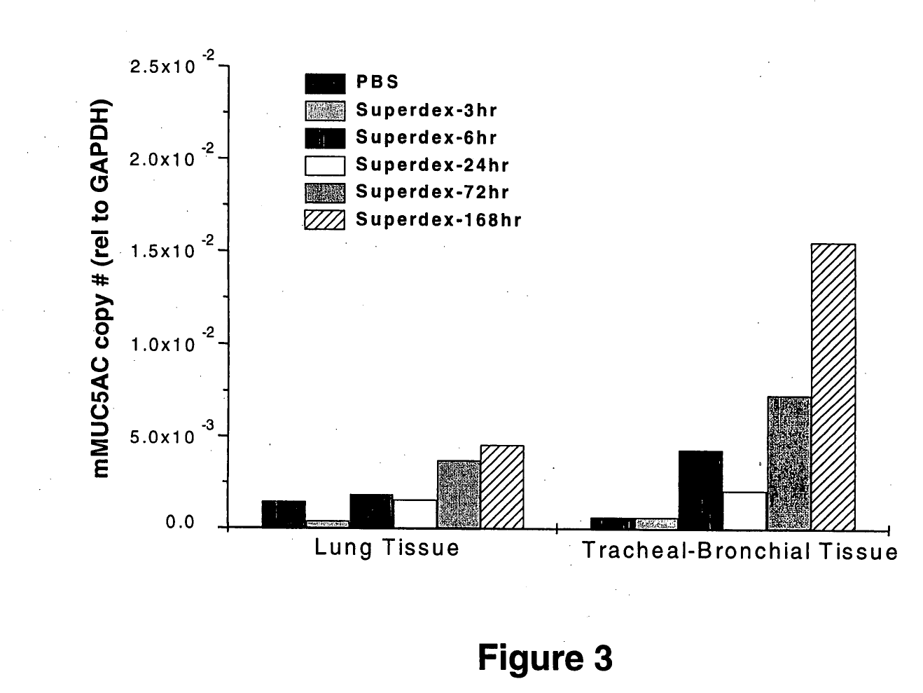

FIG. 4 shows mucin protein release into the lung, following treatment with irritants, as measured by colorometric assay (Alcian Blue/Periodic Acid/Schiff's stain) of the bronchoalveolar lavage.

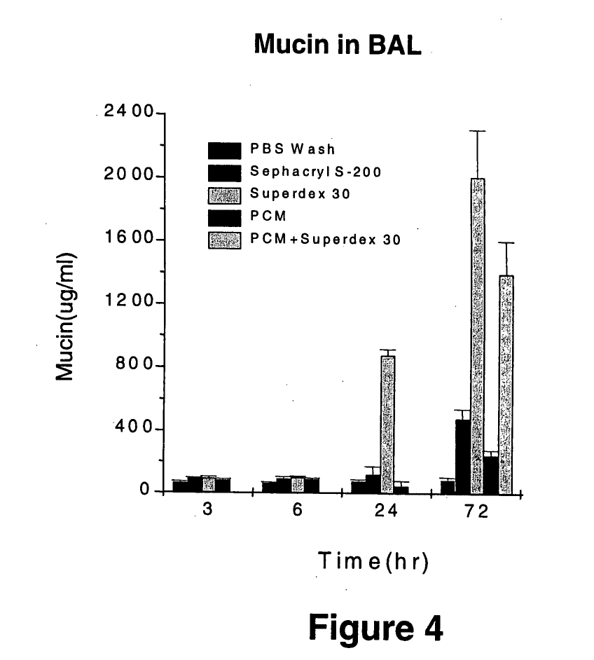

FIG. 5 provides an expression profile for hCLCA1 in normal human tissues.

FIG. 6 provides an expression profile for hMUC2 in normal human tissues.

FIG. 7 provides the expression profile for hCLCA1, 2, 3, and 4 in selected normal human mucosal tissues.

FIG. 8 provides the expression profile for hMUC1, 2, 5AC, 5B, 4, and 6 in selected normal human mucosal tissues.

Definitions

“Ca²⁺-activated Cl⁻ Channel 1” or “CaCC” refer to the amino acid sequences of substantially purified CaCC obtained from any species particularly mammalian species, including bovine, ovine, porcine, murine, equine, and preferably the human species (human CLCA1, hCLCA1), from any source, whether natural, synthetic, semi-synthetic, or recombinant. “CaCC” may refer to the singular, Ca²⁺-activated Cl⁻ Channel, or the plural, Ca²⁺-activated Cl⁻ Channels.

“Agonist” refers to a molecule that modulates the activity of CaCC by increasing or prolonging the duration of the effect of CaCC. Agonists can include nucleotides, proteins, nucleic acids, carbohydrates, organic compounds, inorganic compounds, or any other molecules which modulate the effect of CaCC.

“Amplification” relates to the production of additional copies of a nucleic acid sequence. Amplification can be carried out using polymerase chain reaction (PCR) technologies or other methods well known in the art.

“Antagonist” refers to a molecule which decreases the amount or the duration of the biological or immunological activity of CaCC. Antagonists may include proteins, nucleic acids, carbohydrates, antibodies, organic compounds, inorganic compounds, or any other molecules which exert an effect on CaCC activity.

The term “antisense” refers to any composition containing nucleic acids which is complementary to the “sense” strand of a specific nucleic acid molecule. Antisense molecules may be produced by any method including synthesis or transcription. Once introduced into a cell, the complementary nucleotides combine with natural sequences produced by the cell to form duplexes and to block either transcription or translation.

The designation “negative” can refer to the antisense strand, and the designation “positive” can refer to the sense strand.

A “coding sequence” is a polynucleotide sequence that is transcribed into mRNA and translated into a polypeptide. The boundaries of the coding sequence are determined by a translation start codon at the 5′-terminus and a translation stop codon at the 3′-terminus. A coding sequence can include, but is not limited to, mRNA, cDNA, synthetic DNA, and recombinant polynucleotide sequences. Also included is genomic DNA where the coding sequence is interrupted by introns.

“Complementary” and “complementarity” refer to the natural binding of polynucleotides to other polynucleotides by base pairing. For example, the sequence “5′ A-C-G-T 3′” will bind to the complementary sequence “3′ T-G-C-A 5′.” Complementarity between two single stranded molecules may be “partial,” such that only some of the nucleic acids bind, or it may be “complete,” such that total complementarity exists between the single stranded molecules.

The term “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

The phrase “correlates with expression of a polynucleotid” refers to the detection of the presence of nucleic acids, the same or related to a nucleic acid sequence encoding CaCC, e.g., by northern analysis or RT-PCR, is indicative of the presence of nucleic acids encoding CaCC in a sample, and thereby is indicative of the expression of the transcript from the polynucleotide encoding CaCC.

The phrase “detectably labeled” as used herein means joining, either covalently or non-covalently to the polynucleotides, polypeptides, or antibodies of the present invention, a substance which provides for a detectable signal. A wide variety of labels and conjugation techniques are well known in the art. Suitable labels include radionuclides, e.g., ³²P, ³⁵S, ³H, enzymes, substrates, cofactors, inhibitors, fluorescent moieties, chemiluminescent moieties, magnetic particles, and the like.

The phrase “disease state” means any disease, condition, symptom, disorder, or indication.

The phrase “disease state associated with a mucus secretion disorder”, which is used interchangeably with “symptoms associated with a mucus secretion disorder”, means disease states associated with the gastrointestinal tract, including, but not limited to, inflammatory bowel syndrome, ulcerative colitis, and Crohn syndrome.

The phrases “effective amount” or “therapeutically effective amount” mean a concentration of CaCC modulator sufficient to inhibit or enhance the effect of the CaCC.

The term “expression” as used herein intends both transcriptional and translational processes, i.e., the production of messenger RNA and/or the production of protein therefrom.

The term “modulate” refers to a change in the activity of a CaCC. For example, modulation may cause an increase or a decrease in protein activity, binding characteristics, ion channel opening and conductance, receptor or second messenger signaling, or any other biological, functional, or immunological properties of CaCC. The term “modulator” is a composition having the ability to effect the above changes. The ability to modulate the activity of CaCC can be exploited in assays to screen for organic, inorganic, or biological compounds which affect the above properties of CaCC.

“Nucleic acid” and “nucleic acid sequence” refer to a nucleotide, oligonucleotide, polynucleotide, or any fragment thereof. These phrases also refer to DNA or RNA of genomic or synthetic origin which may be single stranded or double stranded and may represent the sense or the antisense strand, a peptide nucleic acid (PNA), or any DNA-like or RNA-like material. In this context, “fragments” refer to those nucleic acids which, when translated, would produce polypeptides retaining some functional characteristic, e.g., antigenicity, or structural domain, e.g., ion channel domain, characteristic of the full-length polypeptide.

The terms “operably associated” and “operably linked” refer to functionally related but heterologous nucleic acid sequences. A promoter is operably associated or operably linked with a coding sequence if the promoter controls the translation or expression of the encoded polypeptide. While operably associated or operably linked nucleic acid sequences can be contiguous and in the same reading frame, certain genetic elements, e.g., repressor genes, are not contiguously linked to the sequence encoding the polypeptide but still bind to operator sequences that control expression of the polypeptide.

An “oligonucleotide” refers to a nucleic acid molecule of at least about 6 to 50 nucleotides, preferably about 15 to 30 nucleotides, and more preferably 20 to 25 nucleotides, which can be used in PCR amplification or in a hybridization assay. “Oligonucleotide” is substantially equivalent to the terms “amplimer,” “primer,” “oligomer,” and “probe” as these terms are commonly defined in the art.

The term “phenotype” refers to the physical, biochemical, and physiological makeup of an animal as determined both genetically and environmentally.

“Recombinant host cells,” “host cells,” “cells,” “cell lines,” “cell cultures,” and other such terms denoting cell lines cultured as unicellular entities refer to cells which can be, or have been, used as recipients for recombinant vectors or other transfer DNA, immaterial of the method by which the DNA is introduced into the cell or the subsequent disposition of the cell. The terms include the progeny of the original cell which has been transfected. Cells in primary culture as well as cells such as oocytes also can be used as recipients.

A “reporter gene” is a gene that, upon expression, confers a phenotype on a cell expressing the reporter gene, such that the cell can be identified under appropriate conditions. For example, the reporter gene may produce a polypeptide product that can be easily detected or measured in a routine assay. Suitable reporter genes known in the art which confer this characteristic include those that encode chloramphenicol acetyl transferase (CAT activity), β-galactosidase, luciferase, alkaline phosphatase, human growth hormone, fluorescent proteins, such as green fluorescent protein (GFP), and others. Indeed, any gene that encodes a protein or enzyme that can readily be measured, for example, by an immunoassay such as an enzyme-linked immunosorbent assay (ELISA) or by the enzymatic conversion of a substrate into a detectable product, and that is substantially not expressed in the host cells (specific expression with no background) can be used as a reporter gene to test for promoter activity. Other reporter genes for use herein include genes that allow selection of cells based on their ability to thrive in the presence or absence of a chemical or other agent that inhibits an essential cell function. Suitable markers, therefore, include genes coding for proteins which confer drug resistance or sensitivity thereto, or change the antigenic characteristics of those cells expressing the reporter gene when the cells are grown in an appropriate selective medium. For example, reporter genes include: cytotoxic and drug resistance markers, whereby cells are selected by their ability to grow on media containing one or more of the cytotoxins or drugs; auxotrophic markers by which cells are selected by their ability to grow on defined media with or without particular nutrients or supplements; and metabolic markers by which cells are selected for, e.g., their ability to grow on defined media containing the appropriate sugar as the sole carbon source. These and other reporter genes are well known in the art.

A “change in the level of reporter gene product” is shown by comparing expression levels of the reporter gene product in a cell exposed to a candidate compound relative to the levels of reporter gene product expressed in a cell that is not exposed to the test compound and/or to a cell that is exposed to a control compound. The change in level can be determined quantitatively for example, by measurement using a spectrophotometer, spectrofluorometer, luminometer, and the like, and will generally represent a statistically significant increase or decrease in the level from background. However, such a change may also be noted without quantitative measurement simply by, e.g., visualization, such as when the reporter gene is one that confers the ability of cells to form colored colonies on chromogenic substrates, or the ability of cells to thrive and/or die in the presence of test compounds.

“Subject” or “animal” means mammals and non-mammals. Mammals means any member of the Mammalia class including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, reptiles, and the like. The term “subject” does not denote a particular age or sex.

The term “transduction” refers to the genetic recombination in bacteria in which DNA from a donor cell is transferred to a recipient cell by means of a phage. A segment of the donor DNA is first incorporated into the phage DNA, and is then incorporated, by recombination, into the recipient DNA.

The terms “transformed” and “transfected” refer to any known method for the insertion of foreign DNA or RNA sequences into a host cell. Such transformed or transfected cells include stably transformed or transfected cells in which the inserted DNA is rendered capable of replication in the host cell. They also include transiently expressing cells which express the inserted DNA or RNA for limited periods of time. The transformation or transfection procedure depends on the host cell being transformed. It can include packaging the polynucleotide in a virus as well as direct uptake of the polynucleotide, such as, for example, lipofection, electroporation, or microinjection. Transformation and transfection can result in incorporation of the inserted DNA into the genome of the host cell or the maintenance of the inserted DNA within the host cell in plasmid form. Methods of transformation are well known in the art and include, but are not limited to, viral infection, electroporation, lipofection, and calcium phosphate mediated direct uptake.

The term “transgenic animal” or “knockout animal” can refer to an animal engineered to exhibit dysfunctional expression of a target gene or genes.

“Treating” or “treatment of” a disease state includes: 1) preventing the disease state, i.e. causing the clinical symptoms of the disease state not to develop in a subject that may be exposed to or predisposed to the disease state, but does not yet experience or display symptoms of the disease state; 2) inhibiting the disease state, i.e., arresting the development of the disease state or its clinical symptoms; 3) or relieving the disease state, i.e., causing temporary or permanent regression of the disease state or its clinical symptoms.

A “vector” is a replicon in which another polynucleotide segment is attached, such as to bring about the replication and/or expression of the attached segment. The term includes expression vectors, cloning vectors, and the like.

By “intestine” is meant the lower part of the alimentary canal, which extends from the stomach to the anus and is composed of a convoluted upper part (small intestine) and a lower part of greater diameter (large intestine).

By “small intestine” is meant the region of the intestine composed of the duodenum, jejunum, and ileum.

By “large intestine” is meant the region of the intestine composed of the ascending colon, transverse colon, descending colon, sigmoid colon, and rectum.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a CaCC” includes a plurality of such polypeptides and reference to “the assay” includes reference to one or more assays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention is based on the discovery that CaCC, in particular, human CLCA1 (hCLCA1) is causally linked to improper mucus secretion. The causal link was established using a combinatorial ribozyme (RZ) screening assay as described, e.g., in PCT publication, WO 98/50530, published Nov. 12, 1998; and U.S. Ser. No. 60/224,383, filed Aug. 9, 2000, filed herewith, both incorporated herein by reference. Briefly, a human lung epithelial cell line was transfected with a construct comprised of a mucin promoter, MUC5AC, driving a detectable reporter gene, e.g., GFP. The lung epithelial cell line was transduced with a combinatorial RZ library and subsequently stimulated with Pseudomonas aeruginosa broth and PMA to induce mucin gene expression.

Through multiple rounds of sorting, e.g., by FACS, a population of cells lacking MUC5AC promoter (MUC⁻) activation was enriched. The active RZs present in the MUC⁻ cells were PCR amplified, cloned, and sequenced, resulting in Target Sequence Tags (TSTs). Using public and commercially available Expressed Sequence Tag databases, several critical mucin targets were identified, the most notable being human CLCA1 (hCLCA1). This finding implicated hCLCA1 as an important regulator of mucin gene expression and mucus production.

Additionally, expression of the murine homolog of hCLCA1, mGob-5, was correlated to mucin gene expression by studies using an Affymetrix (Santa Clara, Calif.) gene chip studies (Mul 1 k chip) containing mouse lung mRNA isolated from mice treat with IL-13. IL-13 has been shown to induce mucin gene expression in mice when delivered intranasally. (See, e.g., Wills-Karp, et al. (1998) Science 282:22580.) Murine Gob-5 was the strongest induced gene out of 13,500 genes analyzed.

Applying a statistical correlative tool known in the art (Walker, et al. (1999) Genome Res. 9:1198-1203) to the LIFESEQ GOLD database (Incyte Pharmaceuticals, Palo Alto, Calif.), it was shown that one of the mucin genes, MUC2, was coexpressed with hCLCA1. MUC2 is one of the inducible mucin genes in the lung and the intestine, possibly contributing to any one of the nucus hypersecretion or hyposecretion phenotypes described previously.

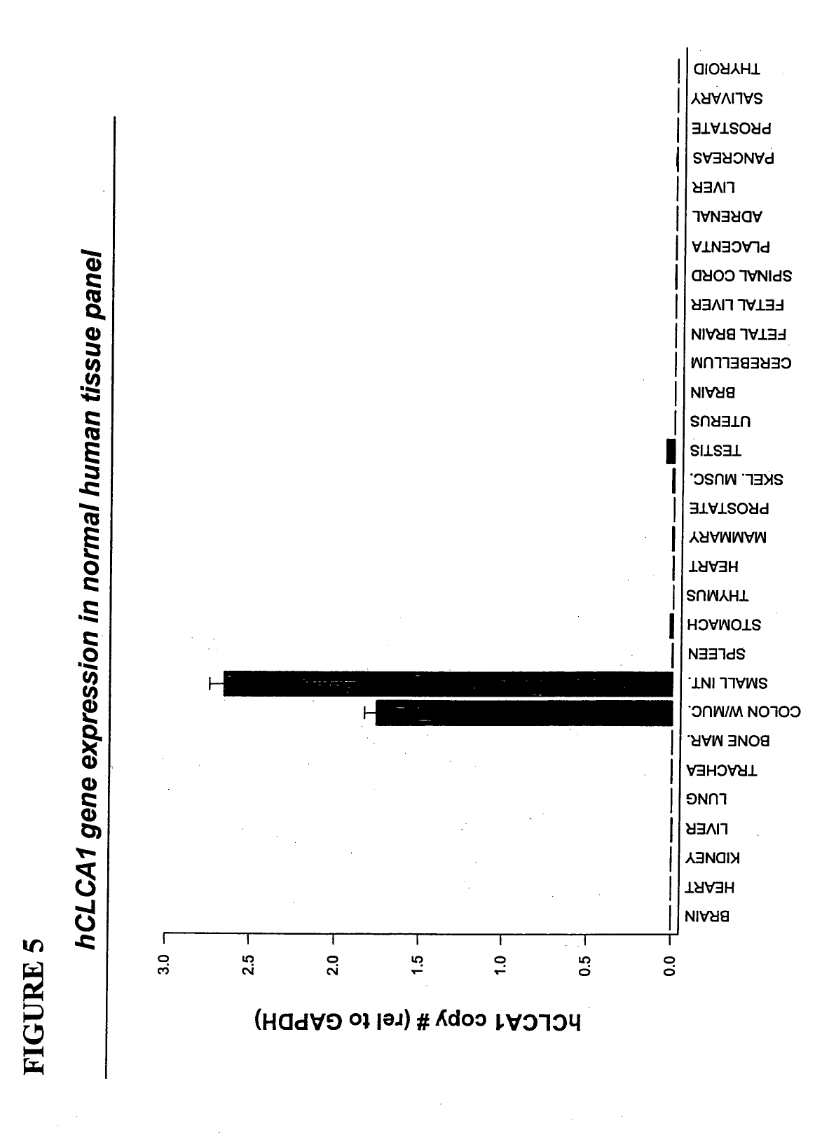

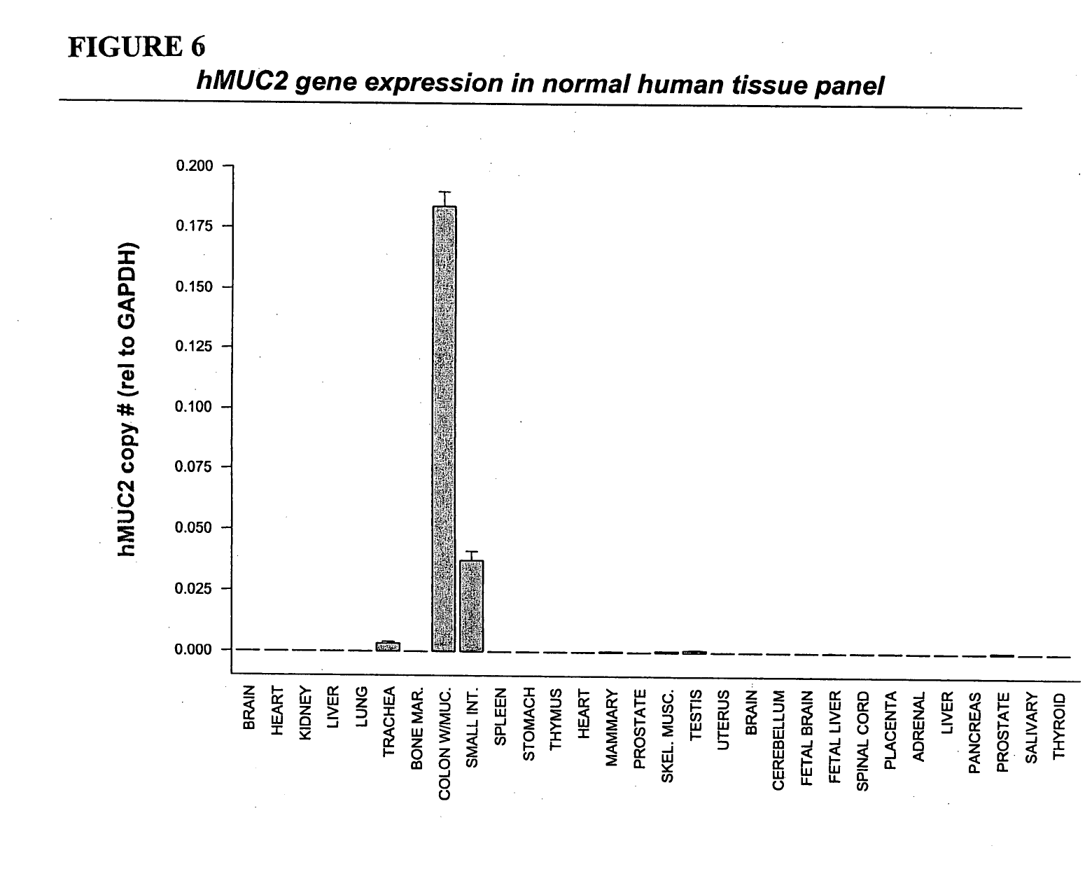

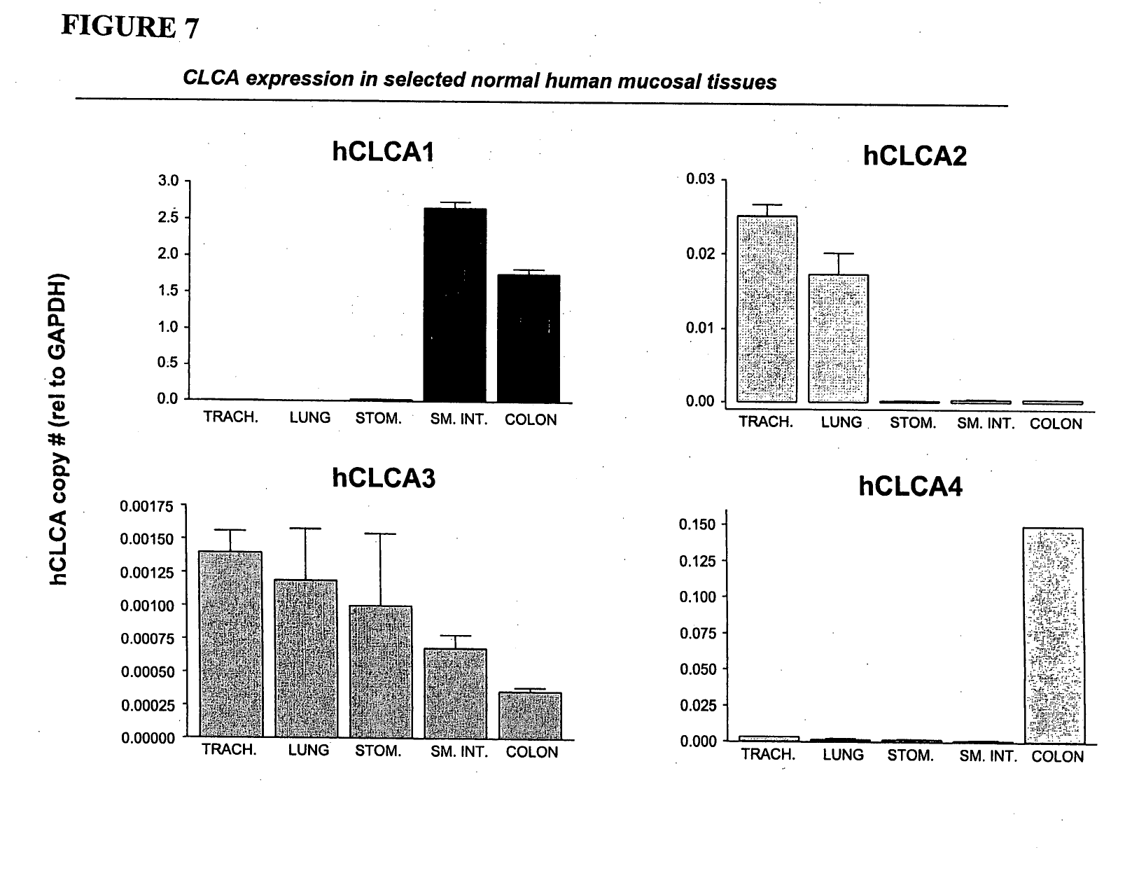

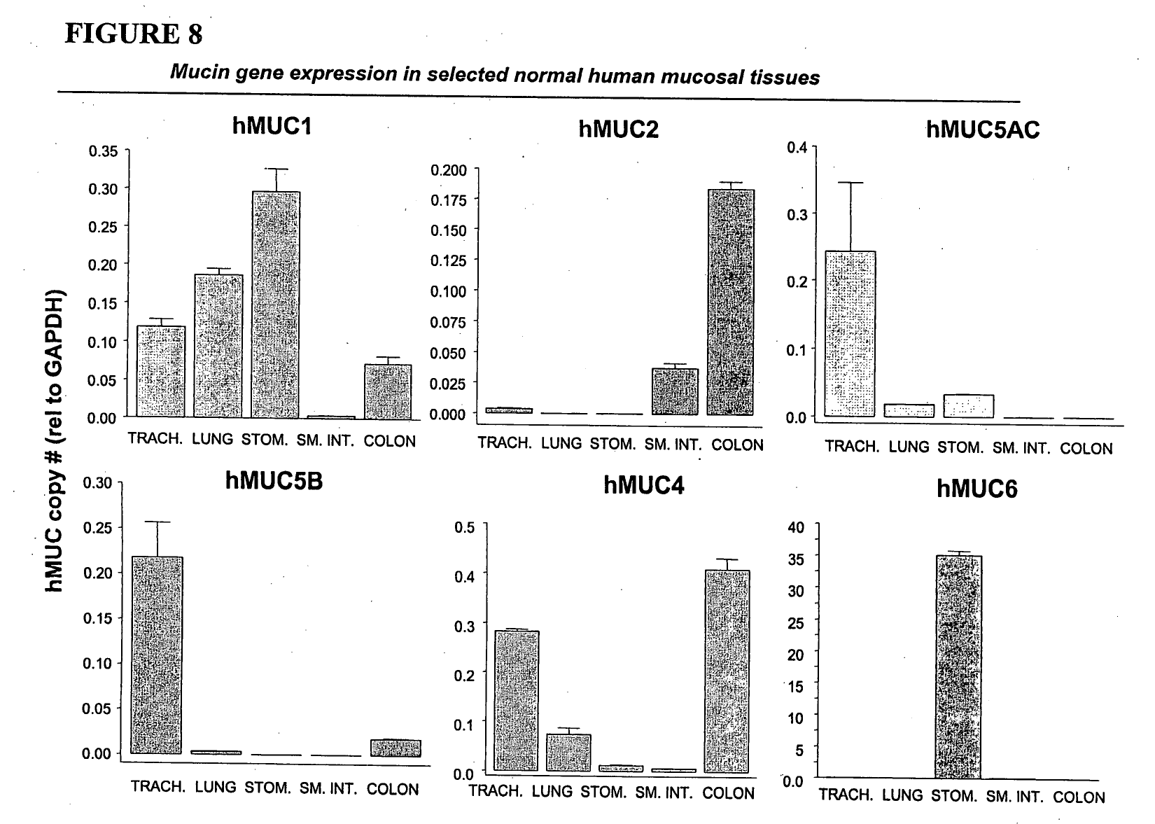

As described in the Examples, hCLCA1 is expressed in the colon and small intestine, while expression of hCLCA1 is very low or undetectable in other tissues tested, including tissues of the respiratory tract (see FIG. 5). The tissue distribution of hCLCA1 expression correlates with that of hMUC2 (see FIG. 6), but not with that of other MUC genes tested (see FIGS. 7 and 8).

In view of the above, modulators of CaCC will be useful in the treatment of disease states associated with mucus secretion disorders. These disorders include, but are not limited to, inflammatory bowel syndrome, ulcerative colitis, and Crohn syndrome.

Methods

The subject invention provides methods of modulating mucin secretion by a gastrointestinal cell. By “gastrointestinal cell” is meant a cell of the gastrointestinal tract of a mammal. Cells of the gastrointestinal tract include mucin-producing cells of the gastrointestinal (GI) tract, and in particular mucin-producing cells of the GI tract. Target cells are cells of the GI tract that normally produce mucin. Such cells may produce higher than normal levels of mucin, or lower than normal levels of mucin in a disease state. In certain embodiments, the target cells are human cells that secrete human mucins, and in particular hMUC2. As shown in Example 6, the tissue distribution of expression of hMUC2 correlates with that of hCLCA1.

As the subject methods are methods of modulating mucin secretion in target cells (e.g., mucin-producing cells of the gastrointestinal tract), they are methods of changing the mucin secretion of target cells, i.e., the amount of mucin secreted by target cells. As such, in certain embodiments the methods are methods of increasing or enhancing mucin secretion by target cells. In other embodiments, the methods are methods of decreasing or inhibiting mucin secretion by target cells.

In practicing the subject methods, the target gastrointestinal tract cell(s) is contacted with an amount of a CLCA1 modulatory agent effective to modulate, e.g., enhance or decrease, mucin secretion by the target cell. By CLCA1 modulatory agent is meant an agent that alters the activity of a CLCA1 protein of the target cell. Target CLCA1 proteins are hCLCA1 and homologs thereof, particularly functional homologs thereof, e.g., murine CLCA, etc. By functional homolog thereof is meant that the homolog has substantially the same mucin secretion modulatory activity, particularly GI tract cell mucin secretion modulatory activity, as hCLCA1. In many embodiments, the subject homologs are proteins whose amino acid sequence is at least about 55%, usually at least about 75% and more usually at least about 90% identical and/or at least about 60% similar, usually at least about 75% and more usually at least about 90% similar over at least a substantial portion of its length, e.g., at least about 50%, usually at least about 75% and more usually at least about 90%, and often at least about 95% and higher, with the amino acid sequence of hCLCA1, and in many embodiments with the sequence of hCLCA1 as reported in Genbank Accession No. XM_{—}030656.

Unless noted otherwise, sequence identity and similarity is determined using Genetics Computer Group (GCG) GAP alignment program (parameters: Gap Weight 8, Length Weight: 2). (GAP was originally written for GCG Version 1.0 by Paul Haeberli from a careful reading of the Needleman and Wunsch (J. Mol. Biol. 48; 443-453 (1970)) and the Smith and Waterman (Adv. Appl. Math. 2; 482-489 (1981)) papers). Specific agents of interest include, but are not limited to: hCLCA1 modulatory agents.

Depending on the desired modulation, the modulatory agents may be agents that enhance the target CLCA1 activity or inhibit the target CLCA1 activity. By target CLCA1 activity is meant activity that affects mucin secretion, e.g., results in enhanced or inhibited mucin secretion.

A wide variety of different types of agents may be employed in the subject methods, including, but not limited to: small organic molecules, nucleic acids, proteins, etc. As such, naturally occurring or synthetic small molecule compounds of interest include numerous chemical classes, though typically they are organic molecules, preferably small organic compounds having a molecular weight of more than 50 and less than about 2,500 daltons. In certain embodiments the agents comprise functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof.

Also of interest as active agents are antibodies that modulate, e.g., inhibit, the target CLCA1 activity in the host. Suitable antibodies are obtained by immunizing a host animal with peptides comprising all or a portion of the target protein, e.g. a CLCA1 protein, such as hCLCA1, etc. Suitable host animals include mouse, rat sheep, goat, hamster, rabbit, etc. The origin of the protein immunogen may be mouse, human, rat, monkey, etc. The host animal will generally be a different species than the immunogen, e.g. human CLCA1 used to immunize mice, etc.

The immunogen may comprise the complete protein, or fragments and derivatives thereof. Preferred immunogens comprise all or a part of a CLCA1, where these residues contain the post-translation modifications, such as glycosylation, found on the native target protein. Immunogens comprising the extracellular domain are produced in a variety of ways known in the art, e.g. expression of cloned genes using conventional recombinant methods, isolation from mucing-producing cells of the GI tract, etc.

For preparation of polyclonal antibodies, the first step is immunization of the host animal with the target protein, where the target protein will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using CLCA1 bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

For in vivo use, particularly for injection into humans, it is desirable to decrease the antigenicity of the antibody. An immune response of a recipient against the blocking agent will potentially decrease the period of time that the therapy is effective. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgGl, IgG3 and IgG4. Either of the human light chain constant regions, kappa or lambda, may be used. The chimeric, humanized antibody is then expressed by conventional methods.

Antibody fragments, such as Fv, F(abN)₂ and Fab may be prepared by cleavage of the intact protein, e.g. by protease or chemical cleavage. Alternatively, a truncated gene is designed. For example, a chimeric gene encoding a portion of the F(abN)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

In yet other embodiments of the invention, the active agent is an agent that modulates, and generally decreases or down regulates, the expression of the gene encoding the target protein in the host. For example, antisense molecules can be used to down-regulate expression of the target CLCA1, e.g., hCLCA1, etc., in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3N—ON-5N—S-phosphorothioate, 3N—S-5N—O-phosphorothioate, 3N-CH₂-5N—O-phosphonate and 3N—NH-5N—O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2N—OH of the ribose sugar may be altered to form 2N—O-methyl or 2N—O-allyl sugars, which provides resistance to degradation without comprising affinity.

Modification of the heterocyclic bases must maintain proper base pairing. Some useful substitutions include deoxyuridine for deoxythymidine; 5-methyl-2N-deoxycytidine and 5-bromo-2N-deoxycytidine for deoxycytidine. 5-propynyl-2N-deoxyuridine and 5-propynyl-2N-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In certain embodiments, the agent is an agent that inhibits the target CLCA1 activity, thereby inhibiting or reducing mucin secretion by the target cell. In these embodiments, the agents are generally CLCA1 antagonists. An agent is considered to be a CLCA1 antagonist if it affects CLCA1 activity in a manner that results in a decrease in mucin secretion by at least about 5%, usually by at least about 10% and more usually by at least about 25%, as compared to a control (e.g. a target cell that has not been contacted with the antagonist). Antagonists of interest include a wide variety of agents, including, but not limited to: small organic molecules, nucleic acids, proteins, etc., where specific types of antagonist agents include those selected from the different types of candidate agents described in greater detail supra and infra with respect to screening assays. CLCA1 antagonists are of interest for treating disorders associated with higher than normal levels of mucus production by cells of the GI tract. Examples of such disorders include ulcerative colitis, and inflammatory bowel syndrome.

Specific inhibitory agents of interest include, but are not limited to: known calcium activated chloride channel (CLCA) inhibitors, such as diisothiocyanatostilbene disulphonic acid (DIDS); dithiothreitol (DTT); niflumic acid (NFA); tamoxifen and the like, (see e.g., Am J Physiol June 1999; 276(6 Pt 1):C1261-70; J Biol Chem Nov. 27, 1998; 273(48):32096-101; and Pflugers Arch May 1998; 435(6):796-803); etc. The agent may be selective for chloride channels, selective for calcium activated chloride channels, selective for specific calcium activated chloride channels, e.g., hCLCA1 or homologs thereof, as may be desired. Agents selective for specific calcium activated chloride channels may be readily identified using the screening assays described supra.

In certain embodiments, the agent is an agent that enhances the target CLCA1 activity, thereby enhancing or increasing mucin secretion by the target cell. In these embodiments, the agents are generally CLCA1 agonists. An agent is considered to be a CLCA1 agonist if it affects CLCA1 activity in a manner that results in an increase in mucin secretion by at least about 5%, usually by at least about 10% and more usually by at least about 25%, as compared to a control (e.g. a target cell that has not been contacted with the antagonist). Agonists of interest include a wide variety of agents, including, but not limited to: small organic molecules, nucleic acids, proteins, etc., where specific types of agonist agents include those selected from the different types of candidate agents described in greater detail supra and infra with respect to screening assays. Agonists are of interest in treating disorders associated with lower than normal levels of mucus secretion by cells of the GI tract. Examples of such disorders include Crohn syndrome.

As mentioned above, the amount of agent that is contacted with the cell is an amount that is effective to cause the desired modulation, e.g., increase or decrease, of mucin secretion from the cell. The amount of change may vary, but is generally at least about 5%, usually at least about 10% and more usually at least about 25% in magnitude. Depending on the nature of the specific modulatory agent, the effective amount may vary, but can be readily determined empirically by those of skill in the art. In many embodiments, the effective amount typically ranges from about 1-500 mg daily, preferably from about 1-100 mg daily, and most preferably from about 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved.

The target cell with which the CLCA1 modulatory agent is contacted during practice of the subject methods may be in an in vitro or in vivo environment, i.e., it may be part of an in vitro or in vivo system. Examples of in vitro environments include cell cultures, etc., while examples of in vivo environments include living organisms, e.g., mammals such as humans, mice, etc.

The CLCA1 modulatory agent is contacted with the target cell(s) using any convenient protocol. The particular protocol employed necessarily depends on the nature of the agent and the environment of the target cell, e.g., whether the target cell is present in an in vitro or in vivo system. For in vitro systems, the modulatory agent is introduced into the in vitro environment of the target cell such that the desired contact occurs, e.g., a composition of the modulatory agent such as an aqueous composition is introduced into a cell culture medium of the target cells, e.g., is pipetted or otherwise dispensed into the culture medium.

Whether a given agent (e.g., CLCA1 antagonist or agonist) acts as a CLCA1 antagonist or agonist can be readily determined using known methods, including, but not limited to, functional assays of the CLCA1 polypeptide (e.g., to determine whether its activity as a calcium activated chloride channel is modulated); immunological assays (e.g., using an antibody specific for a CLCA1 protein, to determine whether protein levels of CLCA1 are affected); RT-PCR assays (e.g., to determine whether expression of the CLCA1 mRNA is affected); assays for mucus production by a mucus-producing cell of the GI tract; and the like.

Methods of Treating Disease Conditions

The subject methods find use in, among other applications, the treatment of disease conditions associated with gastrointestinal tract cell mucin secretion, including GI tract cell mucin hypersecretion and GI tract cell mucin hyposecretion. Disease conditions associated with mucin hypersecretion in the GI tract which are amenable to treatment with the subject methods include, but are not limited to: ulcerative colitis, inflammatory bowel syndrome; and the like. Disease conditions associated with mucin hyposecretion include, but are not limited to: Crohn syndrome; and the like.

By treatment is meant at least an amelioration of the symptoms associated with the mucin secretion associated pathological condition afflicting the host, where amelioration is used in a broad sense to refer to at least a reduction in the magnitude of a parameter, e.g. symptom, associated with the pathological condition being treated, such as excess mucus, insufficient mucus, etc. As such, treatment also includes situations where the pathological condition, or at least symptoms associated therewith, is completely inhibited, e.g. prevented from happening, or stopped, e.g. terminated, such that the host no longer suffers from the pathological condition, or at least the symptoms that characterize the pathological condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Formulations, Dosages, and Routes of Administration

As noted above, the present invention provides a method of treating an animal having disease states associated with mucus secretion disorders as described above. These and other therapeutic uses are described, e.g., in Goodman & Gilman's, (1996) The Pharmacological Basis of Therapeutics, 9^{TH} ed., McGraw-Hill, New York, Chapter 26:601-616; and Coleman, R. A. (1994) Pharmacol. Revs. 46:205-229.

In general, CaCC modulators may be administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able to ascertain a therapeutically effective amount of the compounds of the present invention for a given disease. For example frequency of dosing can be 1, 2, 3, or multiple times per day.

In general, CaCC modulators may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration. An exemplay manner of administration is oral using a convenient daily dosage regimen which can be adjusted according to the degree of affliction. Another exemplary manner of administration is to the gastrointestinal tract via the use of a suppository.

CaCC modulators, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

CaCC modulators may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

CaCC modulators can be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

CaCC modulators may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

CaCC modulators may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

CaCC modulators can be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. [001121 Other suitable pharmaceutical carriers and their formulations are described in Martin (ed.) (1995) Remington: The Science and Practice of Pharmacy, 19th ed., Mack Publishing Company, Easton, Pa.

Screening Assays

The present invention provides various methods for determining whether a compound can modulate the activity of CaCC. The compound can be a substantially pure compound of synthetic origin combined in an aqueous medium, or the compound can be a naturally occurring material such that the assay medium is an extract of biological origin, such as, for example, a plant, animal, or microbial cell extract. The methods essentially entail contacting CaCC or fragments thereof, with the compound under suitable conditions and subsequently determining if the compound modulates the activity of CaCC. The compounds of interest can function as agonists or antagonists of CaCC activity. CaCC or fragments thereof, can be expressed on a cell or tissue, endogenously or recombinantly, or immobilized by attachment to a solid substrate, e.g., nitrocellulose or nylon membrane, glass, beads, etc.

Transcription based assays that identify signals that modulate the activity of cell surface proteins, e.g., receptors, ion channels, etc., may be used to screen candidate compounds for their ability to stimulate reporter gene product expression and their potential to stimulate the expression of CaCC.

One method for identifying compounds that stimulate CaCC promoter-controlled reporter gene expression comprises introducing into a cell a DNA construct that comprises CaCC promoter operably linked to a reporter gene, mixing a test compound with the cell and measuring the level of expression of reporter gene product. A change in the level of expression of the reporter gene product indicates that the compound is capable of modulating the level of CaCC expression. The reporter gene construct is preferably stably integrated into the chromosomal DNA of the cell, but is also functional for the purposes disclosed herein in the form of an extrachromosomal element. The cell may be a eukaryotic cell, or any cell that contains the elements needed to express a structural gene under the regulatory influence of a mammalian gene promoter.

Other transcription based assays are well known in the art. (See, e.g., Zlokarnik, et al. (1998) Science 279:84-88; Siverman, supra; and Gonzalez and Negulescu, (1998) Curr. Opin. Biotechnol. 9:624-631.) These transcription based assays asses the intracellular transduction of an extracellular signal using recombinant cells that are modified by introduction of a reporter gene under the control of a regulatable promoter. In order to express a biologically active CaCC, the nucleotide sequences encoding CaCC or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding CaCC and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview, N.Y.; and Ausubel, F. M. et al. (1989) Current Protocols in Molecular Biology, John Wiley & Sons, New York, N.Y.)

A variety of expression vector/host systems may be utilized to contain and express sequences encoding CaCC. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus) or with bacterial expression vectors (e.g., Ti or pBR322 plasmids); or animal cell systems. The invention is not limited by the host cell employed.

A two-hybrid system-based approach can also be employed for compound screening, small molecule identification, and drug discovery. The underlying premise of the two-hybrid system, originally described in yeast by Fields and Song (1989) Nature 340:245-246, provides a connection between a productive protein-protein or protein-compound interaction pair of interest and a measurable phenotypic change in yeast. A reporter cassette containing an up-stream activation sequence which is recognized by a DNA binding domain, is operationally linked to a reporter gene, which when expressed under the correct conditions will generate a phenotypic change. The original two-hybrid system has recently been modified for applicability in high-throughput compound screening. (See, e.g., Ho et al. (1996) Nature 382:822-826; Licitra and Liu (1996) Proc. Natl. Acad. Sci. USA 93:12817-12821; and Young et al. (1998) Nature Biotech. 16:946-950.)

Assays for identifying compounds that modulate ion channel activity are practiced by measuring the ion channel activity when a cell expressing the ion channel of interest, or fragments thereof, is exposed to a solution containing the test compound and a ion channel selective ion and comparing the measured ion channel activity to the native ion channel activity of the same cell or a substantially identical control cell in a solution not containing the test compound. Methods for practicing such assays are known to those of skill in the art. (See, e.g., Mishina et al. (1985) Nature 313:364-369; and Noda, et al. Nature 322:836-828.)

Ion channel activity can be measured by methods such as electrophysiology (two electrode voltage clamp or single electrode whole cell patch clamp), guanidinium ion flux assays, toxin-binding assays, and Fluorometric Imaging Plate Reader (FLIPR) assays. (See, e.g., Sullivan, et al. (1999) Methods Mol. Biol. 114:125-133; Siegel and Isacoff (1997) Neuron 19:1-20; and Lopatin, et al. (1998) Trends Pharmacol. Sci. 19:395-398.) An “inhibitor” is defined generally as a compound, at a given concentration, that results in greater than 50% decrease in ion channel activity, preferably greater than 70% decrease in ion channel activity, more preferably greater than 90% decrease in ion channel activity.

The binding or interaction of the compound with a target or fragments thereof, can be measured directly by using radioactively labeled compound of interest (see, e.g., Wainscott et al. (1993) Mol. Pharmacol. 43:419-426; and Loric, et al. (1992) FEBS Lett. 312:203-207) or by the second messenger effect resulting from the interaction or binding of the candidate compound. (See, e.g., Lazereno and Birdsall (1993) Br. J. Pharmacol. 109:1120-1127.) Modulation in target signaling or activity can be measured using a detectable assay, e.g., the FLIPR assay. (See, e.g., Coward, P. (1999) Anal. Biochem. 270:242-248; Sittampalam, supra; and Gonzalez and Negulescu, supra.) Activation of certain receptors, in particular, GPCRs, can be measured an ³⁵S-GTP□S binding assay. (See, e.g., Lazareno (1999) Methods Mol. Biol. 106:231-245.)

Alternatively, the candidate compounds can be subjected to competition screening assays, in which a known ligand, preferably labeled with an analytically detectable reagent, most preferably radioactivity, is introduced with the drug to be tested and the capacity of the compound to inhibit or enhance the binding of the labeled ligand is measured. Compounds are screened for their increased affinity and selectivity for the specific receptor or fragments thereof.

Candidate compounds are useful in the treatment or prophylaxis of disease states associated with mucus secretion disorders of the gastrointestinal tract, including, but not limited to, ulcerative colitis, inflammatory bowel syndrome, and Crohn syndrome.

Uses

1. Antisense

The polynucleotides of CaCC can be used to design antisense oligonucleotides that inhibit translation of mRNA encoding CaCC polypeptide. Synthetic oligonucleotides, or other antisense chemical structures are designed to bind to mRNA encoding CaCC and inhibit translation of mRNA and are useful to inhibit expression of CaCC. This invention provides a means to alter levels of expression of CaCC by the use of a synthetic antisense oligonucleotide (SAO) which inhibits translation of mRNA encoding these channels.

SAOs may be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell. (See, e.g., International patent application WO 9523225; and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described, e.g., in Bashkin et al. (1995), Appl. Biochem. Biotechnol. 54:43-56.

The SAO is designed to be capable of passing through cell membranes in order to enter the cytoplasm of the cell by virtue of physical and chemical properties of the SAO which render it capable of passing through cell membranes (e.g. by designing small, hydrophobic SAO chemical structures) or by virtue of specific transport systems in the cell which recognize and transport the SAO into the cell. In addition, the SAO can be designed for administration only to certain selected cell populations by targeting the SAO to be recognized by specific cellular uptake mechanisms which binds and takes up the SAO only within certain selected cell populations. For example, the SAO may be designed to bind to CaCC which are found only in certain cell types.

The SAO is also designed to recognize and selectively bind to the target mRNA sequence by virtue of complementary base pairing to the mRNA. Finally, the SAO is designed to inactivate the target mRNA sequence by any of three mechanisms: 1) binding to the target mRNA and thus inducing degradation of the mRNA by intrinsic cellular mechanisms such as RNAse I digestion; 2) inhibiting translation of the mRNA target by interfering with the binding of translation-regulating factors or of ribosomes; or 3) inclusion of other chemical structures, such as ribozyme sequences or reactive chemical groups, which either degrade or chemically modify the target mRNA.

Synthetic antisense oligonucleotide drugs have been shown to be capable of the properties described above when directed against mRNA targets. (See, e.g., Cohen (1989) Trends in Pharm. Sci. 10:435) In addition, coupling of ribozymes to antisense oligonucleotides is a promising strategy for inactivating target mRNA. (See, e.g., Sarver et al. (1990) Science 247:1222.)

2. Transgenic Animals

Recombinant clones derived from genomic sequences of the homologs of CaCC, e.g., containing introns, will be useful for transgenic and knock-out studies, including transgenic cells, organisms, and knock-out animals, and for gene therapy. (See, e.g., Goodnow (1992) “Transgenic Animals” in Roitt (ed.) Encyclopedia of Immunology, Academic Press, San Diego, Calif., pp. 1502-1504; Travis (1992) Science 254:707-710; Capecchi (1989) Science 244:1288-1292; Robertson (ed.) (1987) Teratocarcinomas and Embryonic Stem Cells: A Practical Approach, IRL Press, Oxford; Rosenberg (1992) J.

Clinical Oncology 10:180-199; Hogan, et al. (eds.) (1994) Manipulating the Mouse Embryo: A Laboratory Manual, 2^{nd} edition, Cold Spring Harbor Press, NY; Wei (1997) Ann. Rev. Pharmacol. Toxicol. 37:119-141; and Rajewsky, et al. (1996) J. Clin. Inves. 98:S51-S53.)

Examples of these techniques include: 1) Insertion of normal or mutant versions of DNA encoding CaCC or homologous animal versions of these genes, e.g., mGob-5, by microinjection, retroviral infection, or other means well known to those skilled in the art, into appropriate fertilized embryos in order to produce a transgenic animal (see, e.g., Hogan, supra); and 2) homologous recombination (see, e.g., Capecchi, supra; and Zimmer and Gruss (1989) Nature 338:150-153) of mutant or normal, human or animal versions of these genes with the native gene locus in transgenic animals to alter the regulation of expression or the structure of CaCC.

The technique of homologous recombination is well known in the art. It replaces the native gene with the inserted gene and is thus useful for producing an animal that cannot express native receptor but does express, for example, an inserted mutant receptor, which has replaced the native receptor in the animal's genome by recombination, resulting in underexpression of the receptor.

Microinjection adds genes to the genome, but does not remove them, and so is useful for producing an animal which expresses its own and added receptors, resulting in overexpression of the receptor. One means available for producing a transgenic animal, with a mouse as an example, is as follows: Female mice are mated, and the resulting fertilized eggs are dissected out of their oviducts. The eggs are stored in an appropriate medium such as M2 medium (see, e.g., Hogan, supra). DNA or cDNA encoding CaCC is purified from an appropriate vector by methods well known in the art. Inducible promoters may be fused with the coding region of the DNA to provide an experimental means to regulate expression of the trans-gene. Alternatively, or in addition, tissue specific regulatory elements may be fused with the coding region to permit tissue-specific expression of the trans-gene. The DNA, in an appropriately buffered solution, is put into a microinjection needle (which may be made from capillary tubing using a pipet puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only method for inserting DNA into the egg cell, and is used here only for exemplary purposes.

Since the normal action of receptor-specific drugs is to activate or to inhibit the receptor, the transgenic animal model systems described above are useful for testing the biological activity of drugs directed against CaCC even before such drugs become available. These animal model systems are useful for predicting or evaluating possible therapeutic applications of drugs which activate or inhibit CaCC by inducing or inhibiting expression of the native or trans-gene and thus increasing or decreasing expression of normal or mutant CaCC in the living animal. Thus, a model system is produced in which the biological activity of drugs directed against CaCC are evaluated before such drugs become available.

The transgenic animals which over or under produce CaCC indicate, by their physiological state, whether over or under production of CaCC is therapeutically useful. It is therefore useful to evaluate drug action based on the transgenic model system. One use is based on the fact that it is well known in the art that a drug such as an antidepressant acts by blocking neurotransmitter uptake, and thereby increases the amount of neurotransmitter in the synaptic cleft. The physiological result of this action is to stimulate the production of less receptor by the affected cells, leading eventually to under expression. Therefore, an animal which under expresses receptor is useful as a test system to investigate whether the actions of such drugs which result in under expression are in fact therapeutic. Another use is that if overexpression is found to lead to abnormalities, then a drug which down-regulates or acts as an antagonist to CaCC is indicated as worth developing, and if a promising therapeutic application is uncovered by these animal model systems, activation or inhibition of CaCC is achieved therapeutically either by producing agonist or antagonist drugs directed against CaCC or by any method which increases or decreases the expression of CaCC in man.

Diagnostics and Kits

The present invention contemplates the use of CaCC polynucleotides, polypeptides, and antibodies in a variety of diagnostic methods kits. Typically the kit will have a compartment containing either a defined CaCC polypeptide, polynucleotide, or a reagent which recognizes one or the other, e.g., antigen fragments or antibodies. Additionally the kit will include the reagents needed to carry out the assay in a separate compartment as well as instructions for use and proper disposal.

A variety of protocols including ELISA, RIA, and FACS for measuring CaCC are known in the art and provide a basis for diagnosing altered or abnormal levels of CaCC expression. These techniques can also be used to monitor the efficacy of therapeutic intervention on the expression of CaCC. For example, an effective therapeutic compound will either inhibit expression of CaCC or induce expression of CaCC. As noted above, a change in the expression level of CaCC can cause a change in MUC5A, thereby affecting the level of mucus secretion.

Normal or standard values for CaCC expression are established by combining body fluids or cell extracts taken from normal mammalian subjects, preferably human, with antibody to CaCC under conditions suitable for complex formation. The amount of standard complex formation may be quantified by various methods, but preferably by photometric means. Quantities of CaCC expressed in control and disease samples from biopsied tissues are compared with the standard values. Deviation between standard and subject values establishes the parameters for diagnosing disease. This method can also be employed to monitor CaCC expression during treatment of a subject with at least one CaCC modulator, in order to evaluate the progress of therapeutic treatment.

In another embodiment of the invention, the polynucleotides encoding CaCC may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of CaCC may be correlated with disease. The diagnostic assay may be used to distinguish between absence, presence, and excess expression of CaCC, and to monitor regulation of CaCC levels during therapeutic intervention, as noted above.

In one aspect, hybridization with PCR probes which are capable of detecting polynucleotide sequences, including genomic sequences, encoding CaCC or closely related molecules, may be used to identify nucleic acid sequences which encode CaCC. The specificity of the probe, whether it is made from a highly specific region, e.g., 10 unique nucleotides in the 5′ regulatory region, or a less specific region, e.g., especially in the 3′coding region, and the stringency of the hybridization or amplification (maximal, high, intermediate, or low) will determine whether the probe identifies only naturally occurring sequences encoding CaCC, alleles, or related sequences.

Probes may also be used for the detection of related sequences, and should preferably contain at least 50% of the nucleotides from any of the CaCC encoding sequences. The hybridization probes of the subject invention may be DNA or RNA, including genomic sequence including promoter, enhancer elements, and introns of the naturally occurring CaCC.

Means for producing specific hybridization probes for DNAs encoding CaCC include the cloning of nucleic acid sequences encoding CaCC or CaCC derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences encoding CaCC may be used for the diagnosis of conditions or disorders which are associated with expression of CaCC, e.g., disease states associated with mucus secretion disorders.

In order to provide a basis for the diagnosis of disease associated with expression of CaCC, a normal or standard profile for expression is established. This may be accomplished by combining body fluids or cell extracts taken from normal subjects, either animal or human, with a polynucleotide sequence, or a fragment thereof, which encodes CaCC, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from subjects who are symptomatic for disease. Deviation between standard and subject values is used to establish the presence of disease.

Once a disease is established and a treatment protocol is initiated, hybridization assays may be repeated on a regular basis to evaluate whether the level of expression of CaCC in the subject begins to approximate that which is observed in the normal subject. The results obtained from successive assays may be used to show the efficacy of treatment over a period ranging from several hours to several days to several months.

Additional diagnostic uses for oligonucleotides designed from the sequences encoding CaCC may involve the use of PCR. Such oligomers may be chemically synthesized, generated enzymatically, or produced in vitro. Oligomers will preferably consist of two nucleotide sequences, one with sense orientation (5′to 3′) and another with antisense (3′to 5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of CaCC include radiolabeling or biotinylating nucleotides, coamplification of a control nucleic acid, and standard curves onto which the experimental results are interpolated. (See, e.g, Melby, P. C. et al. (1993) J. Immunol. Methods, 159:235-244; and Duplaa, C. et al. (1993) Anal. Biochem. 212:229-236.) The speed of quantitation of multiple samples may be accelerated by running the assay in an ELISA format where the oligomer of interest is presented in various dilutions and a spectrophotometric or colorimetric response gives rapid quantitation.

In another embodiment of the invention, the nucleic acid sequences which encode CaCC may also be used to generate hybridization probes which are useful for mapping the naturally occurring genomic sequence. The sequences may be mapped to a particular chromosome, to a specific region of a chromosome or to artificial chromosome constructions, such as human artificial chromosomes (HACs), yeast artificial chromosomes (YACs), bacterial artificial chromosomes (BACs), bacterial P1 constructs, or single chromosome cDNA libraries. (See, e.g., Price, C. M. (1993) Blood Rev. 7:127-134, and Trask, B. J. (1991) Trends Genet. 7:149-154.)

Fluorescent in situ hybridization (FISH) may be correlated with other physical chromosome mapping techniques and genetic map data. (See, e.g., Verma et al. (1988) Human Chromosomes: A Manual of Basic Techniques, Pergamon Press, New York, N.Y.) Examples of genetic map data can be found in various scientific journals or at Online Mendelian Inheritance in Man (OMIM). Correlation between the location of the gene encoding CaCC on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region (see, e.g., Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Some of the standard methods are described or referenced, e.g., in Maniatis et al. (1982) Molecular Cloning: A Laboratory Manual, Cold Spring Harbor Laboratory, Cold Spring Harbor Press; Sambrook et al. (1989) Molecular Cloning: A Laboratory Manual, (2d ed.), vols. 1-3, CSH Press, N.Y.; or Ausubel et al. (1987 and Supplements) Current Protocols in Molecular Biology, Greene/Wiley, New York; Innis et al. (eds.)(1990) PCR Protocols: A Guide to Methods and Applications, Academic Press, N.Y. Methods for protein purification include such methods as ammonium sulfate precipitation, column chromatography, electrophoresis, centrifugation, crystallization, and others. See, e.g., Ausubel et al. (1987 and periodic supplements); Deutscher (1990) “Guide to Protein Purification” in Methods in Enzvmol., vol. 182, and other volumes in this series; and manufacturer's literature on use of protein purification products, e.g., Pharmacia, Piscataway, N. J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering Principle and Methods, Plenum Press, N.Y., 12:87-98; and Crowe et al. (1992) OIAexpress: The High Level Expression & Protein Purification System QIAGEN, Inc., Chatsworth, Calif.

### Example 1

### Construction of Mucin Expressing Cell Line

Using standard techniques (see, e.g., Ausubel, supra) 3.8 Kb of the human MUC5AC promoter sequence (GenBank Accession No. AF016834; and Li, et al. (1998) J. Biol. Chem. 273:6812-6820) was cloned into an expression vector such that transcriptional elements of the MUC5AC promoter drove the expression of a reporter gene, e.g., GFP.

Following stable transfection into a human lung epithelial cell line, NCI-H292 (see, Kai, et al. (1996) Am. J. Physiol. 271 :L484-488) and selection of a stable transfectant (Clone 8), the MUC5AC promoter and expression of the reporter gene was induced by addition of Pseudomnas-conditioned media (PCM). Mucin promoter activation was then quantified, e.g., evaluation of GFP levels by fluorescence microscopy.

### Example 2

### Screening of Mucin Expressing Cell Line with Ribozyme Library

A combinatorial ribozyme library of gene expression inhibitors was synthesized in a retroviral vector system as described in PCT publication, WO 98/50530, published Nov. 12, 1998, and U.S. Ser. No. 60/224,383, filed Aug. 9, 2000, both of which are incorporated herein by reference. Clone 8 was transduced with the ribozyme expressing retrovirus and mucin expression was induced by PCM as described previously.

After 24 hours, the retrovirally transduced Clone 8 cells were separated by FACS based on the level of activation of the MUC5AC promoter. The lowest expressing cells were allowed to recover and subsequently subjected to further rounds of FACS in order to select cells in which mucin expression was reproducibly diminished. By the second and third rounds of FACS, a discernable population of PCM-nonresponders was detected.

### Example 3

### Identification of Genes Causally Linked to Mucin Gene Expression

Sequences of individual combinatorial ribozymes were extracted from PCR-derived DNA sequence using retroviral vector sequence as a marker for the location of the random ribozyme sequence. The mRNA-binding region of each ribozyme was used to search the LifeSeq EST database (Incyte Genomics, Palo Alto, Calif.) using a Smith-Waterman algorithm. Genes that were hit by two independent ribozymes, thus confirming selective inhibition by these genes, were selected as candidate targets. One of the strongest hits was the human CaCC (hCLCA1; GenBank Accession No.

AF039400), which was identified ten times. This gene was placed in a Target Sequence Tags (TST) database which incorporated all expressed genes putatively inhibited by an individual ribozyme.

### Example 4

### Induction of the Mouse Homolog of hCLCA1, mGOB5

To confirm the above combinatorial ribozyme result, the role of CLCA1 in a mouse model of mucin hypersecretion was investigated as described in Lee, et al. (2000) Am. J. Physiol. Lung Cell. Mol. Physiol. 278:L185-192. Quantitative RT-PCR assays (Taqman assays, PE Biosystems) were used to assess the levels of mCLCA1 (GenBank Accession No. AF047838); mGOB5 (GenBank Accession No. ABO1756); and MUC5AC (GenBank Accession No. L42292). GAPDH was used as a housekeeping gene.

In the mouse model of mucus hypersecretion, mGOB5 (the homolog of hCLCA1) was strongly induced by superdex bead instillation. Maximum expression was detected at 3 and 7 days after treatment. Higher expression levels were detected in the trachiobronchial tissue when compared to the rest of the lung. In contrast mCLCA1, which is a homolog of human CLCA3, was much lower than mGOB5, and not induced.

### Example 5

### Correlation of MUC5AC Gene Expression with mGOB5/hCLCA1 Gene Expression

Further analysis of the quantitative RT-PCR (Taqman) results indicated a strong correlation of mouse MUC5AC (the mouse homolog of human MUC5AC) with mGOB5. This correlative relationship was confirmed by detection of mature mucin protein secretion into the lumen of the lung. Bronchoalveolar lavage of superdex treated mouse lung from the above study showed mucin release as measured by colorometric assay (Alcian Blue/Periodic Acid/Schiff's stain). Mucin protein release increased after bead instillation, which correlates with the expression of mGOB5 and MUC5AC, but not mCLCA1.

### Example 6

### Correlation of hMUC2 Gene Expression with hCLCA1 Expression in Normal Human Gastrointestinal Tissue

Analysis of the expression of hCLCA1 in various normal human tissues was conducted using the primer pairs shown in Table 1, and RT-PCR. The protocol for this assay is described in the user manual for the Taqman Gold RT-PCR kit, sold by Perkin Elmer Biosystems. Using literature sequences as references, standard curves of each cloned gene were created to allow us to accurately quantify both relative and absolute levels of CLCA gene expression (mRNA levels) in our normal human tissues. We obtained tissues from a commercial supplier (Clontech); these were typically pooled samples taken from trauma victims (i.e., car accidents) who were presumed healthy at the time of death. PCR products were analyzed using the probes shown in Table 1.

hCLCA1 expression in a wide variety of normal human tissues was examined. As shown in FIG. 5, hCLCA1 is expressed in normal human colon and small intestine. Only background levels of expression were detected in the other tissues tested. As shown in FIG. 6, the tissue distribution of hCLCA1 expression correlates with that of hMUC2.

The tissue distribution of hCLCA1 and hMUC2 expression was compared with that of other CLCA and MUC genes. For these analyses, mRNA from trachea, lung, stomach, small intestine, and colon was analyzed. As shown in FIG. 7 and 8, the tissue distribution of hCLCA1 expression correlates with that of hMUC2, but not with that of HMUC1, 5AC, 5B, 4, or 6. Whereas hMUC5AC and hCLCA2 are both expressed in respiratory tissue, hCLCA1 and hMUC2 are both expressed in the small intestine and colon, but not in the trachea or lung.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

TABLE I

| Gene | GenBank | | | |
|---|---|---|---|---|
| name | Accession # | Forward Primer | Reverse Primer | Taqman Probe |
| hGAPDH | M33197 | GTTCGACAGTCAGCCGCATC | GGAATTTGCCATGGGTGGA | ACCAGGCGCCCAATACGACCAA |
| | | SEQ ID NO:1 | SEQ ID NO:2 | SEQ ID NO:3 |
| hMUC1 | AF084521 | GCCAGGATCTGTGGTGGTACA | CTCCACGTCGTGGACATTGA | GGCACATCACTCACGCTGACGTCTGA |
| | | SEQ ID NO:4 | SEQ ID NO:5 | SEQ ID NO:6 |
| hMUC2 | L21998 | CCCAACTTTGATGCCAGCATT | CAGCATCCATTGGGCATGA | TGTGATGGAGCCCGGGATGCA |
| | | SEQ ID NO:7 | SEQ ID NO:8 | SEQ ID NO:9 |
| hMUC4 | AJ010901 | CGAAAACAGCCCACTGATGTC | TGGAGGCCTGAGTTGGAATT | AGGGCGATACCTCTCCCACACTGGC |
| | | SEQ ID NO:10 | SEQ ID NO:11 | SEQ ID NO:12 |
| hMUC5 | ACU06711 | TACTCGCTCGAGGGCAACA | TGCAGTGCAGGGTCACATTC | CCAGGAGCTGCGGACCTCGC |
| | | SEQ ID NO:13 | SEQ ID NO:14 | SEQ ID NO:15 |
| hMUC5B | Z72496 | GTGTGGGTGGTCTCTGGAGTAGA | AAATCCACAGCTACCAGCTTTACA | TGGACCGTCCCAGCACAGACCA |
| | | SEQ ID NO:16 | SEQ ID NO:17 | SEQ ID NO:18 |
| hMUC6 | HSU97698 | CCACTTCTGCCTCCATCCA | GGCCTTGAGCGTTGTTGGT | TCAACGCCAACAGGCACCGTTC |
| | | SEQ ID NO:19 | SEQ ID NO:20 | SEQ ID NO:21 |
| hCLCA1 | AF039400 | GCAAGGTGGCTTTGTAGTGGA | AGACTGTATTTCCAAGTGCCAACC | ACACCAAAATGGCCTACCTCCAAATCCC |
| | | SEQ ID NO:22 | SEQ ID NO:23 | SEQ ID NO:24 |
| hCLCA2 | AF043977 | GAGGCCGAGTGTTTGTCCAT | CCATTTATGTAGAAAGGTTTGTCATTG | TGGGCCCACCTCCGTTGGG |
| | | SEQ ID NO:25 | SEQ ID NO:26 | SEQ ID NO:27 |
| hCLCA3 | AF043976 | CCTGAAGTCACAGATGATGTGGAA | AGGCACTCCTGATACAGTAAACGA | CAGACGACTTCAGCAGACTCACCTCTGG |
| | | SEQ ID NO:28 | SEQ ID NO:29 | SEQ ID NO:30 |
| hCLCA4 | AF127035 | GAATCAAGCAGCAAAACATTTCC | GTGGCAGTACTATCAAAGTGAACCA | CCCAGGATCCATTTTCAACAGTCTGCAG |
| | | SEQ ID NO:31 | SEQ ID NO:32 | SEQ ID NO:33 |
| mGAPDH | M32599 | GTCCCGTAGACAAAATGGTGAAG | GTGACCAGGCGCCCAAT | CGGTGTGAACGGATTTGGCCG |
| | | SEQ ID NO:34 | SEQ ID NO:35 | SEQ ID NO:36 |

1. A method of treating a subject having a disease state associated with a mucus secretion disorder comprising administering to the subject an effective amount of a hCLCA1 modulator.

2. The method of claim 1, wherein the animal is a mammal.

3. The method of claim 2, wherein the mammal is a human.

4. The method of claim 1, wherein the disease state is associated with mucus hypersecretion.

5. The method of claim 4, wherein the disease state associated with mucus hypersecretion is selected from the group consisting of ulcerative colitis and inflammatory bowel syndrome.

6-9. (canceled)

10. The method of claim 1, wherein the hCLCA1 modulator is an antagonist.

11-22. (canceled)

23. The method of claim 1 wherein said hCLCA1 modulator comprises a ribozyme.

24. A method of modulating the mucus secretion of a target cell, comprising:
(a) providing a mammalian cell capable of mucus secretion that expresses hCLCA1; and
(b) contacting said mammalian cell with an effective amount of a hCLCA1 modulator.

25. The method of claim 24, wherein said contacting comprises in vivo administration.
